SEQUENCE LISTING (10 sequences)

SEQ ID NO: 1 - DNA, 21 bp, Artificial Sequence
  Primer
taaacatgaaaactcgttccg

SEQ ID NO: 2 - DNA, 26 bp, Artificial Sequence
  Primer
ttttatgatgagaacacttaaactca

SEQ ID NO: 3 - DNA, 43 bp, Artificial Sequence
  Primer
accgcatcgaatcgatgtaaaatagaaaatcgcatgcaagata

SEQ ID NO: 4 - DNA, 47 bp, Artificial Sequence
  Primer
tatcgattccgctccagacttaaaaagctgcctcagaatatactcag

SEQ ID NO: 5 - DNA, 28 bp, Artificial Sequence
  Probe
gctttgttaggtaaagctctgatatttg

SEQ ID NO: 6 - DNA, 20 bp, Artificial Sequence
  Primer
atgcggttccacgatcttgg

SEQ ID NO: 7 - DNA, 20 bp, Artificial Sequence
  Primer
gcgactgctgttgaatcacc

SEQ ID NO: 8 - DNA, 44 bp, Artificial Sequence
  Primer
ccaattcacaagtgaagagcaaaatctcctgcccgaattcgtaa

SEQ ID NO: 9 - DNA, 44 bp, Artificial Sequence
  Primer
tctaccgctgatcatgtgctaaaatgctcagctgtattagcctc

SEQ ID NO: 10 - DNA, 28 bp, Artificial Sequence
  Probe
gcccgaattcgtaaatgatgatggcgtc

(19) United States
(12) Patent Application Publication
(10) Pub. No.: US 2015/0079587 A1
(43) Pub. Date: Mar. 19, 2015

(54) METHOD FOR DETECTING NUCLEIC ACIDS BY SIMULTANEOUS ISOTHERMAL AMPLIFICATION OF NUCLEIC ACIDS AND SIGNAL PROBE

(71) Applicants: Min Hwan KIM, Seongnam-si (KR); Sook LEE, Ansan-si (KR); Un Ok KIM, Gyeongju-si (KR); Ji Won JEONG, Seoul (KR); Joo Hee LEE, Gwangju-si (KR)

(72) Inventors: Min Hwan KIM, Seongnam-si (KR); Sook LEE, Ansan-si (KR); Un Ok KIM, Gyeongju-si (KR); Ji Won JEONG, Seoul (KR); Joo Hee LEE, Gwangju-si (KR)

(73) Assignee: Green Cross Medical Science Corp., Yongin-si (KR)

(21) Appl. No.: 13/894,400

(22) Filed: May 14, 2013

Related U.S. Application Data
(63) Continuation-in-part of application No. 12/745,544, filed on May 30, 2010, now abandoned.

(30) Foreign Application Priority Data
Dec. 3, 2007 (KR) 10-2007-0124399

(51) Int. Cl. C12Q 1/68

(52) U.S. Cl. CPC C12Q 1/6853
USPC 435/6.11

(57) ABSTRACT

Method for detecting target nucleic acids by simultaneous isothermal amplification of the target nucleic acids and a signal probe 5 using an external primer set, a DNA-RNA-DNA hybrid primer set and a DNA-RNA-DNA hybrid signal probe. The method can be used to amplify target nucleic acids in a sample, rapid and exact manner without the risk of contamination compared to the conventional methods such as PCR, and it can simultaneously amplify target nucleic acid and a signal probe, so that it can be applied to various genome projects, detection and identification of a pathogen, detection of gene modification producing a predetermined phenotype, detection of hereditary diseases or determination of sensibility to diseases, and estimation of gene expression. Thus, the method is useful for molecular biological studies and disease diagnosis.

### CROSS-REFERENCE

This application is a continuation-in-part application of U.S. patent application Ser. No. 12/745,544 filed on May 30, 2010, based on International Application PCT/KR2008/002341 filed on Apr. 24, 2008 entitled “METHOD FOR DETECTING NUCLEIC ACIDS BY SIMULTANEOUS ISOTHERMAL AMPLIFICATION OF NUCLEIC ACIDS AND SIGNAL PROBE”, claiming a priority of Korean Patent Application No. 10-2007-0124399 filed on Dec. 3, 2007, all of which are incorporated herein by reference in their entireties.

### TECHNICAL FIELD

An embodiment of the present invention relates to a method for isothermal amplification of nucleic acids and a signal probe, and a method for detecting target nucleic acids by isothermal amplification of signal probe. More particularly, an embodiment of the present invention relates to a method for detecting target nucleic acids rapidly by simultaneously amplifying target nucleic acids and a single probe using an external primer set, a DNA-RNA-DNA hybrid primer set and a DNA-RNA-DNA hybrid signal probe.

### BACKGROUND ART

A nucleic acids amplification technique is very useful for detecting and analyzing a small quantity of nucleic acid. A high sensibility to target nucleic acids in the nucleic acids amplification enables to develop a technology of detecting specific nucleic acids in a field of gene separation for diagnosis and analysis of infectious disease and genetic disease and in medicolegal field. Based on such method for detecting nucleic acid, the various methods which can execute a very sensitive diagnosis and analysis have been developed (Belkum, Current Opinion in Pharmacology, 3:497, 2003). Detection of nucleic acid is achieved by complementarily of DNA strands and the ability of single stranded nucleic acid to form double stranded hybrid molecules in vitro. Due to this ability, it is possible to detect specific nucleic acids in a sample (Barry et al, Current Opinion in Biotechnology, 12:21, 2001).

A probe used in detection of nucleic acid is composed of specific sequences capable of hybridize with a target sequence present in a nucleic acid sample. The probe is read by chemical materials, immune chemicals, fluorescent materials or radioisotopes. Generally, probes are composed to include fluorescent materials capable of reading DNA hybridization and fragmentary nucleic acids having complementary sequence to target nucleic acids, or markers or report molecules such as biotin and digoxygenin.

However, the above mentioned methods have problems in that they cannot detect a short sequence on the chromosomal DNA, result in low copy numbers and has a difficulty to solve the problem of the limited copy number of modified allele of wild-type gene. Another problem of the method is related to in vitro or in situ environmental conditions, which limit physical interaction among a target sequence, a chemicals, a probe and an another molecular structures.

The method for detection of target nucleic acid is classified into three categories, that is, (1) target sequence amplification in which target nucleic acids are amplified, (2) probe amplification in which a probe molecule itself is amplified, and (3) signal amplification in which each probe signal is increased by probe hybridization technique or multiplex ligation-dependent probe amplification technique.

In vitro nucleic acid amplification techniques have been used as leading methods in detecting and analyzing a small quantity of nucleic acid. Among them, PCR (polymerase chain reaction) is most widely used as a nucleic acid amplification technique which uses repeated cycles of primer-dependent nucleic acid synthesis occurring simultaneously using each strand of a complementary sequence as a template and thus copies of each strand of a complementary sequence are synthesized. However, in order to carry out PCR, a pre-programmed thermal cycling instrument is needed. Also, PCR technique has the following shortcomings: it costs a lot; it has a relatively low specificity; performance procedure should be extremely standardized to reproduce RCR results.

In LCR (ligase chain reaction) which is another nucleic acid amplification technique, two neighboring oligonucleotides are hybridized with target nucleic acids, and ligased with a ligase, and then a probe formed through this method is amplified by temperature cycling together with a complementary nucleotide.

Since LCR has higher discriminatory power than primer extension using a primer, it shows higher allele specificity than that of PCR in genotyping point mutation. Among nucleic acid amplification techniques developed up until now, LCR has the highest specificity and it is the easiest method to perform because all of discrimination mechanisms are optimized. However, it has a shortcoming in that its reaction rate is the slowest and it requires many modified probes.

In methods using ligation such as LCR, genotyping can be performed by amplifying a primarily circularized padlock probe through DNA ligation accompanied by process of LCR or RCA (rolling circle replication), using RCA technique without PCR target amplification (Qi et al, Nucleic Acids Res., 29:e116, 2001)

However, the amplification method using heat cycle process such as PCR requires a heat block to reach “target” temperature of each cycle, and a delay time until the heat block reaches the target temperature, therefore it takes a long time until the amplification reaction is completed.

SDA (strand displacement amplification) is an amplification method of a target nucleic acid sequence and the complementary strand thereof in samples by strand displacement using endonuclease. This method uses a mixture containing nucleic acid polymerase, at least one primer complementary to 3′-terminal end of a target fragment and dNTPs (deoxynucleoside triphosphates) comprising at least one substituted dNTP. Each primer has a sequence in 5′-terminal end, which restriction endonuclease can recognize (Walker et al, Nucleic Acids Res., 29:1691, 19921.

As similar methods to SDA, there are SPIA (single primer isothermal amplification) technique using 5′-RNA-DNA-3′ primer (U.S. Pat. No. 5,251,639), ICAN (isothermal chimeric primer-initiated amplification of nucleic acid) technique using 5′-DNA-RNA-3′ primer (US 2005/0123950) and Ribo primer technique, using RNA primer (US 2004/0180361) etc, in which after an extension of a primer using an RNA-DNA hybrid primer or an RNA primer, a primer and a template DNA is digested with RNaseH digesting an RNA primer hybridized with a template DNA, and then a new primer is extended by strand displacement.

TMA (transcription mediated amplification) is a target nucleic acid amplification technique using only one promoter-primer at a constant temperature, a constant ionic strength and a constant pH (Kwoh et al, Proc. Nat. Acad. Sci. USA, 86:1173, 1989). TMA comprises the step of combining a mixture composed of target nucleic acids and promoter-primer which is an oligonucleotide complementary to the 3′-terminal end of a target sequence for hybridization with the 3′-terminal of target nucleic acids or neighboring region thereof. The promoter-primer comprises a sequence of promoter region for RNA polymerase located in the 5′-terminal end of 0 complexing sequence. The promoter-primer and target sequence form a promoter-primer/target sequence hybrid to extend DNA.

In the process of DNA extension of TMA technique, it is assumed that the 3′-terminal end of a target sequence is extended from the location close to a complex in which a promoter-primer is hybridized between a complexing sequence and a target sequence. A promoter sequence produces a first DNA extension product to act as a template in an extension process forming a double stranded promoter sequence. The 3′-terminal end of the promoter-primer could be used as a primer in the second DNA extension process. In the extension process, a double stranded 0 nucleic acid complex is formed using a target sequence as a template. When a RNA target sequence is used, the complex is a DNA/RNA complex and when a DNA target sequence is used, the complex is a DNA/DNA complex. Subsequently, an RNA polymerase recognizing a promoter of the promoter-primer synthesizes RNA using the first DNA extension product in order to produce various RNA copies of target sequence.

NASBA (nucleic acid sequence-based amplification) technique comprises syntheses of single stranded RNA, single stranded DNA and double stranded DNA (Compton, Nature, 350:91, 1991). The single stranded RNA becomes the first template for the first primer, the single stranded DNA becomes the second template for the second primer, and the double stranded DNA becomes the third template in synthesis of copies for the first template.

Since the method for isothermal amplification of target nucleic acids such as SDA, NASBA and TMA is performed at a constant temperature, it does not require a separate thermal cycling apparatus, and thus, it is easy to perform. However, the isothermal amplification methods of target nucleic acids disclosed up until now have several disadvantages. The method according to SDA requires a specific region for a given restriction enzyme, so the application thereof is limited. The transcription-based amplification methods such NASBA and TMA require the binding between a polymerase promoter sequence and an amplification product by a primer, and this process tends to bring a non-specific amplification. Because of these disadvantages, the amplification mechanism of DNA target by transcription-based amplification methods has not been well-established.

Moreover, currently used amplification methods are disadvantageous in that there is a possibility of test samples being contaminated by the products of preceding amplification reaction, thereby causing non-specific target amplification. In order to prevent this, contamination detection methods of a sample solution which employ various means including physical means for decontaminating the sample in the last step of amplification reaction or before the beginning of target nucleic acid amplification, are being developed, but most of them make nucleic acid amplification procedure complicated.

A method for amplifying a probe as another method for detecting nucleic acids include LCR method used in said nucleic acid amplification methods.

As another method for detecting nucleic acid, there is a method for amplifying a signal, not a target nucleic acid and a probe. Among these methods, there is bDNA (branched DNA) amplification method using four sets of probes to capture a target nucleic acid (Ross et al, J. Viral. Method., 101:159, 2002). Hybrid capture method using signal amplification has sensitivity comparable to the method for directly detecting and amplifying a target nucleic acid, and uses an antibody or a luminous chemical for signal detection (van der Pol et al, J. Clinical Microbiol, 40:3564, 2002; Nelson et al, Nucleic Acids Research, 24:4998, 1996).

Furthermore, there is CPT (cycling probe technology) as a method for amplifying a signal probe (Duck et al, Biotechniques, 9: 142, 1990). The method uses a DNA/RNA/DNA hybrid probe having a base sequence complementary to a target nucleic acid. In the method, a signal probe is amplified by repeating a procedure, in which when a signal probe is hybridized with a target nucleic acid, RNA region of the hybrid signal probe is digested with RNaseH and the digested hybrid signal probe is separated from the target nucleic acid, then another DNA-RNA-DNA hybrid probe is hybridized with the target nucleic acid. However, the CPT (cycling 5 probe technology) method has disadvantages in that it has a relatively low amplification efficiency of 10²˜10⁴, so it is difficult to be used independently in diagnosis, and the process thereof is complicated, and high cost and long processing time is required, since the signal probe is separately amplified after a special region of a target nucleic acid is amplified by conventional nucleic acid 0 amplification such as PCR.

U.S. Pat. No. 5,824,517 (Cleuziat et al.) discloses an isothermal amplification method using an external primer and a DNA-RNA-DNA hybrid primer set, but it does not the use of a DNA-RNA-DNA hybrid signal probe. Also, US 2005/0214809 (Han et al.) discloses the use of a DNA-RNA-DNA hybrid signal probe in the detection of isothermally amplified nucleic acids that is about labeling modification of cycling probe technology (CPT) probe (Bekkaoui et al. Diagn. Microbiol. Infect. Dis. 34: 83, 1999), but it does not mention a specific length of bases or a favorable effect due to the same.

Meanwhile, the present inventors have developed a method for detecting target nucleic acids by simultaneous isothermal amplification of nucleic acids and a signal probe using a RNA-DNA hybrid primer, etc. (Korean patent Publication No, 10-2006-0085818). However, the method has disadvantages in that cost of hybridization is high since RNA-DNA hybrid primer has RNA region of 15˜25 bases and thus the cost of RNA monomers is high, and the stability of the hybrid primer may be increased upon purification and storage thereof due to the chemical characteristic of RNA highly susceptible to hydrolysis compare to DNA.

### SUMMARY

An aspect of the present invention is to provide a method for amplifying a target nucleic acid and a signal probe at isothermal temperature rapidly and exactly.

Another aspect of the present invention is to provide a method for detecting target nucleic acids, which comprises performing simultaneous isothermal amplification of target nucleic acids and probe signals.

To achieve the above aspects, an embodiment of the present invention provides a method for isothermal amplification of target DNA, the method comprising the steps of:
(a) denaturing a reaction mixture containing (i) target DNA, (ii) an external primer set having a base sequence complementary to the target DNA, and (iii) a DNA-RNA-DNA hybrid primer set having a base sequence complementary to the target DNA at the 3′-terminal end and non-complementary to the target DNA at the 5′-terminal end, wherein the DNA-RNA-DNA hybrid primer set consists of 44˜66 bases in length, the 5′-DNA region of the DNA-RNA-DNA hybrid primer is 20˜30 basis in length, the RNA region of the DNA-RNA-DNA hybrid primer is 4˜6 bases and the 3′-DNA of the DNA-RNA-DNA hybrid primer is 20˜30 bases in length; and
(b) adding an enzymatic reaction mixture solution containing RNase, DNA polymerase capable of performing, strand displacement and a DNA-RNA-DNA hybrid signal probe having a base sequence complementary to the amplification product produced by the external primer set and the hybrid primer set, to the reaction mixture denatured in the step (a), wherein the DNA-RNA-DNA hybrid signal probe consists of 24˜36 bases in length and the RNA portion located in the middle thereof consists of 4˜6 bases in length, and then simultaneously amplifying said target DNA and said signal probe at isothermal temperature.

An embodiment of the present invention also provides a method for detecting target DNA, which comprises using the amplified signal probe.

An embodiment of the present invention also provides a method for isothermal amplification of target RNA, the method comprising the steps of:
adding a reaction mixture containing (i) target RNA, (ii) an external primer set having a base sequence complementary to the target RNA, and (iii) a DNA-RNA-DNA hybrid primer set having a base sequence complementary to the target RNA at the 3′-terminal end and non-complementary to the target RNA at the 5-terminal end, wherein the DNA-RNA-DNA hybrid primer set consists of 44˜66 bases in length, the 5′-DNA region of the DNA-RNA-DNA hybrid primer is 20˜30 basis in length, the RNA region of the DNA-RNA-DNA hybrid primer is 4˜6 bases and the 3′-DNA of the DNA-RNA-DNA hybrid primer is 20˜30 bases in length, to an enzymatic reaction mixture solution containing (iv) DNA polymerase capable of performing strand displacement, RNase, reverse transcriptase and a DNA-RNA-DNA hybrid signal probe having a base sequence complementary to the amplification product produced by the external primer set and the hybrid primer set, wherein the DNA-RNA-DNA hybrid signal probe consists of 24˜36 bases in length and the RNA portion located in the middle thereof consists of 4˜6 bases in length, and then simultaneously amplifying said target RNA and said signal probe at isothermal temperature.

An embodiment of the present invention also provides a method for detecting target RNA, which comprises using the amplified signal probe.

Other features and aspects of the present invention will be apparent from the following detailed description and the appended claims.

### BRIEF DESCRIPTION OF DRAWINGS

FIG. 1 is a schematic figure of the method for isothermal amplification of target DNA according to an embodiment of the present invention.

FIG. 2 is a schematic figure of the method for isothermal amplification of target DNA and a signal probe according to an embodiment of the present invention.

FIG. 3 is a schematic figure of the method for isothermal amplification of target RNA according to an embodiment of the present invention.

FIG. 4 is a schematic figure of the method for isothermal amplification of target RNA and a signal probe according to an embodiment of the present invention.

FIG. 5 is an electrophoresis photograph of amplification products produced by the method for isothermal amplification of target DNA according to an embodiment of the present invention

FIG. 6 is a schematic diagram of a process for detecting a signal probe produced by the amplification method according to an embodiment of the present invention, by means of enzyme-immunoassay.

FIG. 7 is an analysis result of detecting a signal probe produced by the target DNA amplification method according to an embodiment of the present invention, by means of enzyme-immunoassay.

FIG. 8 is a schematic diagram of a process for detecting a signal probe produced by the target DNA amplification method according to an embodiment of the present invention, by means of lateral-flow chromatography.

FIG. 9 is an analysis result of detecting a signal probe produced by the target DNA amplification method according to an embodiment of the present invention, by means of lateral-flow chromatography.

FIG. 10 is an electrophoresis photograph of amplification products produced by the method for isothermal amplification of target RNA according to an embodiment of the present invention

FIG. 11 is an electrophoresis photograph of amplification products produced by the method for isothermal amplification of target RNA according to an embodiment of the present invention

FIG. 12 is an analysis result of detecting a signal probe produced by the target RNA amplification method according to an embodiment of the present invention, by means of enzyme-immunoassay.

### DETAILED DESCRIPTION

In view of the above, the present inventors have made extensive efforts in order to overcome the problems described above and develop a method for amplifying target nucleic acids in a rapid and exact manner, and at the same time, a method for detecting the amplification product, and as a result, confirmed that when an external primer set having a base sequence complementary to target nucleic acids and a DNA-RNA-DNA hybrid primer set having a base sequence partially complementary to target nucleic acids are used, it is possible to amplify the target nucleic acids rapidly at isothermal temperature while minimizing an RNA region constituting the hybrid primer, and when a DNA-RNA-DNA hybrid probe having a base sequence complementary to the amplification product amplified by the above method is used, it is possible to simultaneously amplify target nucleic acids and probe signals at isothermal temperature, thereby completing embodiments of the present invention.

In one aspect, an embodiment of the present invention relates to a method for isothermal amplification of target DNA, the method comprising the steps of: (a) denaturing a reaction mixture containing (i) target DNA, (ii) an external primer set having a base sequence complementary to the target DNA, and (iii) a DNA-RNA-DNA hybrid primer set having a base sequence complementary to the target DNA at the 3′-terminal end and non-complementary to the target DNA at the 5′-terminal end; and (b) adding an enzymatic reaction mixture solution containing RNase, DNA polymerase capable of performing strand displacement and a DNA-RNA-DNA hybrid signal probe having a base sequence complementary to the amplification product produced by the external primer set and the hybrid primer set, to the reaction mixture denatured in the step (a), and then simultaneously amplifying said target DNA and said signal probe at isothermal temperature.

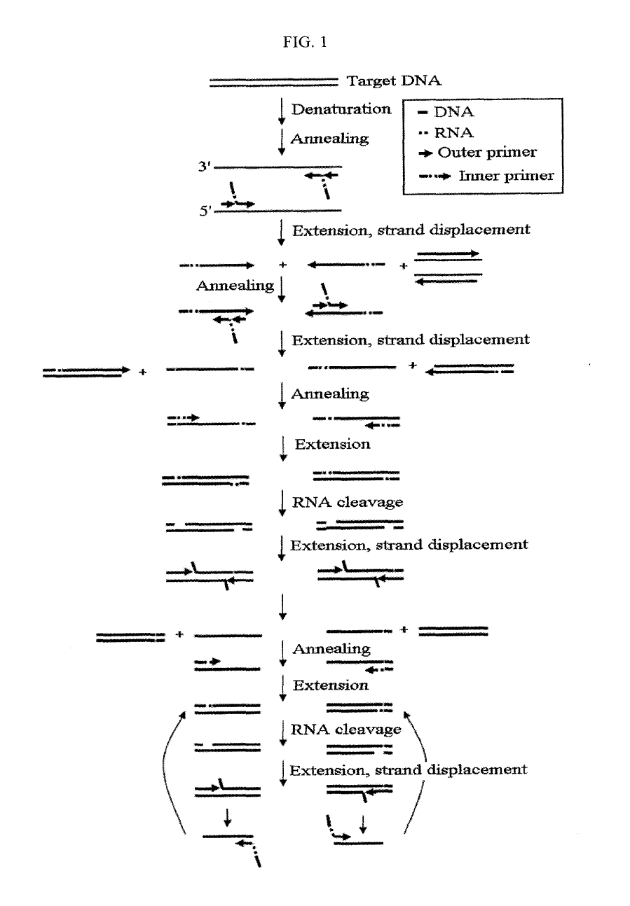

The isothermal amplification of target DNA according to an embodiment of the present invention is carried out in the following manner as shown in FIG. 1. A mixture of target DNA to be amplified as a template in amplification, an external primer set and a DNA-RNA-DNA hybrid primer set is first denatured to render each of them single stranded. The denatured mixture is cooled to isothermal amplification temperature, and an enzymatic reaction mixture solution containing RNase and DNA polymerase is added thereto. The external primer set and DNA-RNA-DNA hybrid primer set are then annealed to the target DNA in the reaction solution cooled to amplification temperature. Preferably, the external primer set comprises a sequence complementary to a sequence closer to both ends of the target DNA than the hybrid primer set, and the hybrid primer set comprises a sequence closer to the middle of the target DNA than the external primer set. In this case, the hybrid primer is annealed in the forward direction of DNA strand extension compared with the external primer. The annealed external primer and hybrid primer are extended using a DNA polymerase capable of performing strand displacement. As the external primer is extended along target DNA, DNA strand extended from the hybrid primer located in the forward direction of extension is separated from target DNA to result in a strand displacement. Finally, single stranded DNA amplification product extended from the hybrid primer and double stranded DNA amplification product extended from the external primer, respectively, are obtained.

The external primer set and hybrid primer set are annealed using single stranded DNA amplification product as a template. The annealed external primer and hybrid primer are extended by a DNA polymerase capable of performing strand displacement, and as the external primer is extended along a single stranded DNA template, a DNA strand extended from the hybrid primer located in the forward direction of extension is separated from a single stranded DNA to result in strand displacement. Finally, single stranded DNA amplification product extended from the hybrid primer and double stranded DNA amplification product extended from the external primer are obtained. The external primer is extended to form a double stranded DNA, the extended DNA-RNA-DNA hybrid primer is separated by strand displacement to obtain a single stranded DNA. The DNA-RNA-DNA hybrid primer is annealed and extended using the amplified single stranded DNA as a template to obtain a double stranded DNA amplification product containing RNA. The RNA region of the double stranded DNA is digested by RNase H, and a single stranded DNA is obtained by strand displacement. Annealing, extension, strand displacement and RNA digestion process is repeated using the single stranded DNA as a template to amplify the target DNA (FIG. 1).

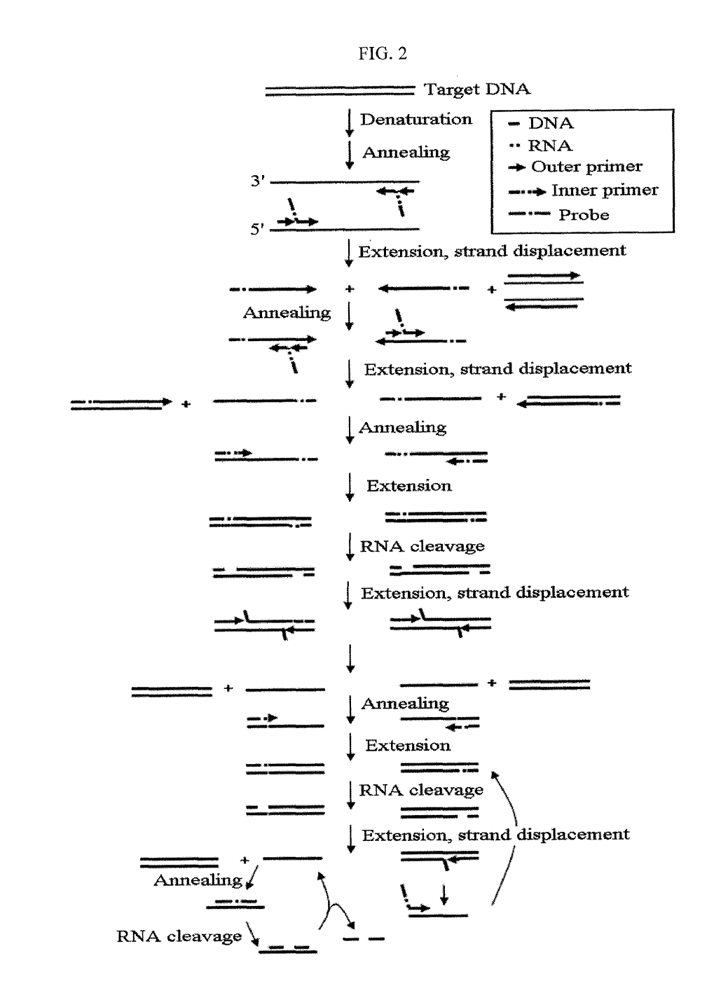

According to one embodiment of the present invention, amplification of a probe signal is simultaneously performed with isothermal amplification of the nucleic acids. After a target DNA amplified by isothermal amplification of the target DNA is annealed with a DNA-RNA-DNA hybrid signal probe to form a double stranded RNA/DNA hybrid, the RNA region of the DNA-RNA-DNA hybrid probe is digested by RNase H activity. Then, the digested signal probe is separated from the target DNA, followed by the binding of a new DNA-RNA-DNA hybrid signal probe to be digested with RNase H and separated. The above described process is repeated to amplify the probe signal (FIG. 2).

It may be essential that the isothermal amplification according to an embodiment of the present invention is conducted by using both two different sites and two kinds of complementary primers (inner & outer). That is, in order to accomplish an isothermal amplification, it is absolutely necessary to use both two different sites and two kinds of complementary primers. Further, 3′-downstream region (DNA) of DNA-RNA-DNA primer must be complementary to a template and 5′-upstream region (DNA-RNA) must be non-complementary to the template.

In an embodiment of the present invention, the external primer set can be any one selected from the group consisting of oligo DNA, oligo RNA, and hybrid oligo RNA/DNA. The external primer set is preferably complementary to the sequence of a target nucleic acid, and preferably has 20˜30 bases in length since the preferable isothermal reaction temperature range is 60˜70° C. otherwise the external primer cannot be annealed to the target DNA or RNA preferably. A target DNA sequence complementary to the external primer is preferably a sequence neighboring a target DNA sequence complementary to a hybrid primer (base difference is 1˜60 bp) and the target DNA sequence complementary to the external primer is preferably a sequence closer to the 3′-end of the target nucleic acid than a target DNA sequence complementary to a hybrid primer.

The DNA-RNA-DNA hybrid primer set used in an embodiment of the present invention is non-complementary to a target DNA at the 5′-end of DNA-RNA, and complementary to the target DNA at the 3′-end of DNA. The DNA-RNA-DNA hybrid primer preferably consists of 44˜66 bases in length, and preferably, the 5′-DNA region of the DNA-RNA-DNA hybrid primer is 20˜30 bases in length since the preferable isothermal reaction temperature range is 60˜70° C. otherwise the 5′-DNA region of the DNA-RNA-DNA hybrid primer cannot be annealed to the target DNA preferably after cleavaging the RNA region. The RNA region of the DNA-RNA-DNA hybrid primer is 4˜6 bases in length. The 3′-DNA region of the DNA-RNA-DNA hybrid primer which is complementary sequence to the target DNA is 20-30 bases in length since the preferable isothermal reaction temperature, range is 60˜70° C. otherwise the 3′-DNA region of the DNA-RNA-DNA hybrid primer cannot be annealed to the target DNA or RNA preferably.

In an embodiment of the present invention, a target DNA sequence complementary to a DNA-RNA-DNA hybrid primer preferably has a sequence closer to the 5-end of a target DNA than a target DNA sequence complementary to an external primer, and a target DNA sequence complementary to a hybrid primer is preferably a sequence neighboring a target DNA sequence complementary to an external primer (base difference is 1˜60 bp).

The DNA polymerase used in an embodiment of the present invention is an enzyme that can extend a nucleic acid primer along a DNA template, and should be capable of displacing a nucleic acid strand from polynucleotide strands. DNA polymerase that can be used in an embodiment of the present invention is preferably a thermostable DNA polymerase with no exonuclease activity and examples thereof include list DNA polymerase, exo(−) vent DNA polymerase, exo(−) Deep vent DNA polymerase, exo(−) Pfu DNA polymerase, Bca DNA polymerase phi29 DNA polymerase etc.

The RNase used in an embodiment of the present invention specifically digests the RNA strand of an RNA/DNA hybrid, and it is preferable not to degrade a single stranded RNA, and RNase H is preferably used.

It is preferable that the DNA-RNA-DNA hybrid signal probe used in an embodiment of the present invention is an oligonucleotide having a sequence complementary to a nucleic acid amplification products amplified by the external primer and DNA-RNA-DNA hybrid primer, and the 5′-end and 3′-end of the DNA-RNA-DNA hybrid signal probe consist of oligo DNA and the middle thereof consists of oligo RNA.

Preferably, the DNA-RNA-DNA hybrid signal probe consists of 24˜36 bases in length since the preferable isothermal reaction temperature range is 60˜70° C. otherwise the DNA-RNA DNA hybrid signal probe cannot be annealed to the nucleic acid amplification products. The RNA portion located in the middle consists of 4˜6 bases in length.

In an embodiment of the present invention, the DNA-RNA-DNA hybrid signal probe is preferably labeled with a marker at an end, and the marker includes biotin, fluorescein, digoxygenin, 2,4-dinitrophenyl and the like.

In an embodiment of the present invention, the isothermal amplification reaction is preferably performed at a temperature at which the inventive primer can be annealed to the DNA template, and the activity of an enzyme used is not substantially inhibited. In an embodiment of the present invention, the amplification temperature is preferably 30-75° C. more preferably 37-70° C., most preferably 60˜70° C.

Moreover, the inventive method for thermal amplification of nucleic acids has high specificity, since it uses an additional external primer compared with conventional methods in which a single RNA-DNA hybrid primer is used (U.S. Pat. No. 6,251,639). Besides, it is possible to significantly improve amplification efficiency by exponential amplification using an inner primer substituted by an external primer as a new template. Moreover, the conventional method uses a separate blocker for blocking amplification or a template-switch oligonucleotide (TSO) to amplify a specific region upon amplification of target base sequences using a single RNA-DNA hybrid primer, on the contrary, the inventive method has an advantage in that only a desired region can be clearly amplified using a forward/reverse primer pair without using a separate blocker or TSO.

The inventive method has an advantage in that it can simultaneously perform amplification and detection of nucleic acids since amplification of nucleic acids and a signal probe can be simultaneously completed in a single-tube by repeating a process, in which a DNA-RNA-DNA hybrid signal probe is bound and separated, using an amplified DNA as a template to amplify the signal probe.

The inventive method also has an advantage in that it does not need to consider problems occurring when reaction activity of RNase is higher than primer extension activity of DNA polymerase in the conventional method, because the 5′-end of DNA-RNA region of the DNA-RNA-DNA hybrid primer used in the present invention, has a sequence non-complementary to a template.

The inventive isothermal amplification of nucleic acids, when a newly synthesized amplification product is used as a new template after a first primer extension and strand displacement reaction, the RNA region non-complementary to the template acts as a template complementary to a primer to increase the annealing temperature for the primer, thus improving amplification efficiency, as well as, preventing primer-dime formation to enhance purity of amplification product.

The method for isothermal amplification of nucleic acids according to an embodiment of the present invention requires about 1 hr achieve complete amplification, starting from DNA extraction in a sample, if DNA extraction was already completed, it requires about 40 min, thereby making it is possible to perform rapid amplification.

In another aspect, an embodiment of the present invention relates to a method for detecting target DNA, the method comprising the steps of: (a) denaturing a reaction mixture containing (i) target DNA, (ii) an external primer set having a base sequence complementary to the target DNA, and (iii) a DNA-RNA-DNA hybrid primer set having a base sequence complementary to the target DNA at the 3′-terminal end and non-complementary to the target DNA at the 5′-terminal end; (b) adding an enzymatic reaction mixture solution containing RNase, DNA polymerase capable of performing strand displacement and a DNA-RNA-DNA hybrid signal probe having a base sequence complementary to the amplification product produced by the external primer sot and the hybrid primer set, to the reaction mixture denatured in the step (a), and then simultaneously amplifying said target DNA and said signal probe at isothermal temperature; and (c) detecting the target DNA from the target DNA amplification product and signal probe amplification product amplified in the step (b) using enzyme-immunoassay or lateral flow chromatography.

The signal probe amplified according to the method of an embodiment of the present invention can be detected using horseradish peroxidase in a microplate (Bekkaoui et al, Diagn. Microbial. Infect. Dis., 34:83-93, 1999). In this case, the DNA-RNA-DNA hybrid probe is preferably end-labeled with fluorescein and biotin, respectively. In the signal probe amplification, the signal probe can be detected by, but not limited to, the following procedure: the signal probe is bound to a microwell plate surface treated with streptavidin binding selectively to biotin, and HRP (horseradish peroxidase) conjugated with anti-fluorescein antibody binding selectively to fluorescein, and washed, then allowed to react with TMB (tetranitrobenzidine) substrate for HRP, followed by measuring the absorbance change at 465 nm. Also, a marker conjugated with an antibody binding selectively to 2,4-dinitrophenyl or digoxygenin, in addition to fluorescein and biotin, can be used.

Also, the signal probe amplified according to an embodiment of the present invention can be detected on a nitrocellulose membrane using lateral flow assay (Fong et al, J. Clin. Microbiol, 38:2525-2529, 2000). In this case, the DNA-RNA-DNA hybrid signal probe is preferably end-labeled with fluorescein and biotin, respectively. In the signal probe amplification, the signal probe can be detected visibly on a nitrocellulose membrane by, but not limited to, binding signal probe to a gold material conjugated with streptavidin binding selectively to biotin and a strip surface-treated with fluorescein antibody binding selectively to fluorescein. Also, a marker conjugated with an antibody binding selectively to 2,4-dinitrophenyl or digoxygenin, in addition to fluorescein and biotin, can be used.

In still another aspect, an embodiment of the present invention relates to a method for isothermal amplification of target RNA, the method comprising the steps of: adding a reaction mixture containing (i) target RNA, (ii) an external primer set having a base sequence complementary to the target RNA, and (iii) a DNA-RNA-DNA hybrid primer set having a base sequence complementary to the target RNA at the 3′-terminal end and non-complementary to the target RNA at the 5′-terminal end; to an enzymatic reaction mixture solution containing (iv) DNA polymerase capable of performing strand displacement, RNase, reverse transcriptase and a DNA-RNA-DNA hybrid signal probe having a base sequence complementary to the amplification product produced by the external primer set and the hybrid primer set, and then simultaneously amplifying said target RNA and said signal probe at isothermal temperature.

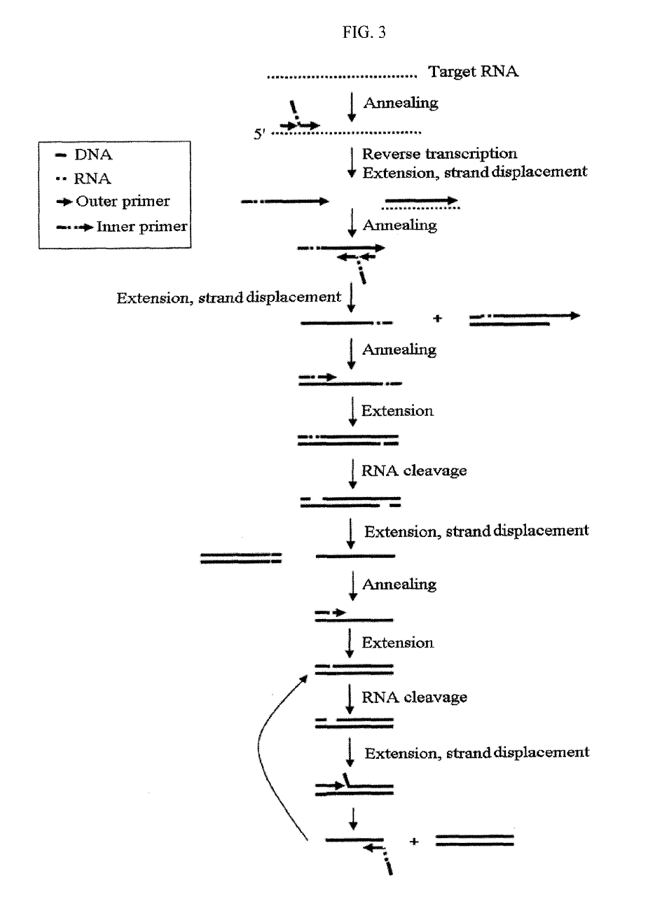

As shown in FIG. 3, isothermal amplification of target RNA according to an embodiment of the present invention is carried out in the following manner: a DNA-RNA-DNA hybrid signal probe is added with a target RNA as a template, an external primer set, a DNA-RNA-DNA hybrid primer set, and an enzymatic reaction mixture solution containing DNA polymerase, RNase, and reverse transcriptase. The external primer set and DNA-RNA-DNA hybrid primer set are then annealed to the target RNA in the reaction solution to amplification temperature. Preferably, the external primer set comprises a sequence complementary to a sequence closer to both ends of the target RNA than the hybrid primer set, and the hybrid primer set comprises a sequence closer to the middle of the target RNA than the external primer set. In this case, the hybrid primer is annealed in the forward direction of DNA strand extension compared with the external primer. Finally, a single stranded DNA amplification product extended from the hybrid primer and a double stranded DNA amplification product, DNA/RNA hybrid, are obtained.

The external primer set and hybrid primer set are annealed using single stranded DNA amplification product as a template. The annealed external primer and hybrid primer are extended by a DNA polymerase capable of performing strand displacement, and as the external primer is extended along a single stranded DNA template, a DNA strand extended from the hybrid primer located in the forward direction of extension is separated from the target DNA to result in strand displacement. Finally, single stranded DNA amplification product extended from the hybrid primer and double stranded DNA amplification product extended from the external primer are obtained. The external primer is extended to form a double stranded DNA, the extended DNA-RNA-DNA hybrid primer is separated by strand displacement to obtain a single stranded DNA. The DNA-RNA-DNA hybrid primer is annealed and extended using the amplified single stranded DNA as a template to obtain a double stranded DNA amplification product containing RNA. The RNA region of the double stranded DNA is digested by RNase H, and a single stranded DNA is obtained by strand displacement. Annealing, extension, strand displacement and RNA digestion process is repeated using the single stranded DNA as a template to amplify the target RNA (FIG. 3).

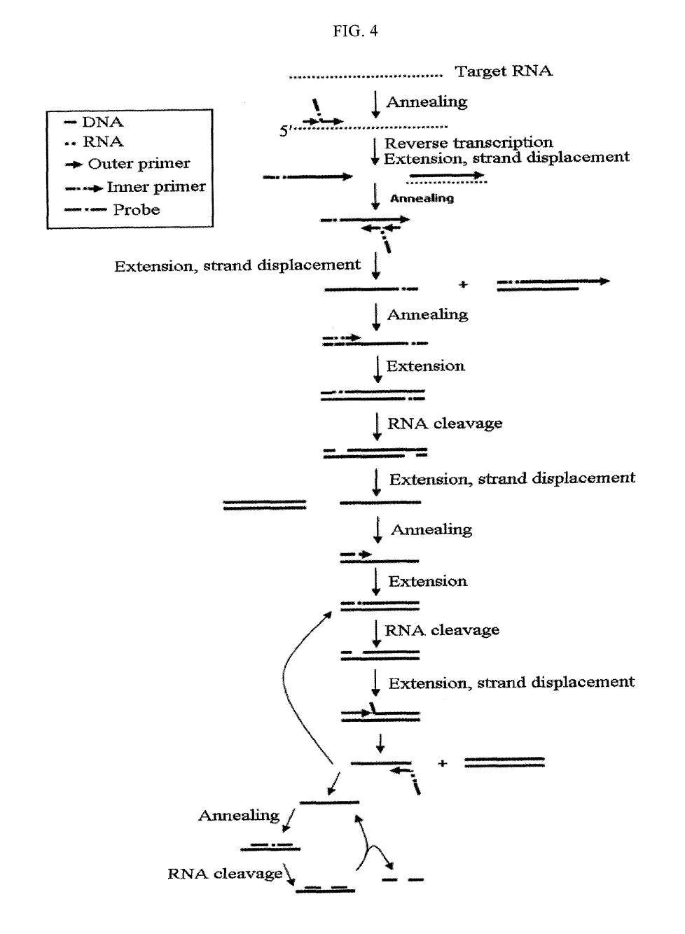

According to another embodiment of the present invention, amplification of a probe signal is simultaneously performed with isothermal amplification of a target RNA. After the target DNA amplified by isothermal amplification of the target RNA is annealed with a DNA-RNA-DNA hybrid signal probe to form a double stranded RNA/DNA hybrid, the RNA region of the DNA-RNA-DNA hybrid probe is digested by RNase H activity. Then, the digested signal probe is separated from the target DNA, followed by the binding of a new DNA-RNA-DNA hybrid probe to be digested with RNase H and separated. The above described cycle is repeated to amplify the probe signal (FIG. 4).

The isothermal amplification of target RNA according to the present invention, except reverse transcriptase additionally added to the enzymatic reaction mixture solution, an external primer set, a DNA-RNA-DNA hybrid primer set, DNA polymerase, RNase, a DNA-RNA-DNA hybrid primer and a DNA-RNA-DNA hybrid signal probe can be used in the above mentioned isothermal amplification of target DNA. Also the isothermal amplification can be performed at the same amplification temperature as that of isothermal amplification of target DNA. The reverse transcriptase is used to extend DNA using RNA as a template and AMV (Avian Myeloblastosis Virus) reverse transcriptase or MMLV (Maloney Murine Leukemia Virus) reverse transcriptase is preferably used.

In yet another aspect, an embodiment of the present invention relates to a method for detecting target RNA, the method comprising the steps of: adding a reaction mixture containing (i) a target RNA, (ii) an external primer set having a base sequence complementary to the target RNA, and (iii) a DNA-RNA-DNA hybrid primer set having a base sequence complementary to the target RNA at the 3′-terminal end and non-complementary to the target RNA at the 5′-terminal end to an enzymatic reaction mixture solution containing (iv) DNA polymerase capable of performing strand displacement, RNase, reverse transcriptase and a DNA-RNA-DNA hybrid signal probe having a base sequence complementary to the amplification product produced by the external primer set and the hybrid primer set, and then simultaneously amplifying said target RNA and said signal probe at isothermal temperature; and detecting the target DNA from the target RNA amplification product and signal probe amplification product amplified in the above step using enzyme-immunoassay or lateral flow chromatography.

The inventive isothermal amplification method and detection method of nucleic acids (DNA, RNA) can amplify in a rapid and simple manner since it employs one-step method in which the reaction is carried out at a constant temperature, and thus it does not require a separate heat transducer due to isothermal amplification of target nucleic acids and a signal probe. Additionally, the method exactly amplifies only target nucleic acids by using two pairs of primers and a probe compared with conventional methods, as well as, amplifies the signal probe, thereby having excellent specificity.

The inventive isothermal amplification method and detection method of nucleic acids is carried out in one tube and thus it is possible to treat in large quantities for real-time detection of nucleic acids. Such advantage can minimize the risk of an additional reaction by contamination which limits a wide use of amplification technique.

### EXAMPLES

Hereinafter, an embodiment of the present invention will be described in more detail by examples. However, it is obvious to a person skilled in the art that these examples are for illustrative purposes only and are not construed to limit the scope of the present invention.

### Example 1

### Isothermal Amplification of DNA

Chlamydia trachomatis (ATCC VR-887) DNA was used as target nucleic acids. Genomic DNA was extracted from Chlamydia trachomatis which is gram negative bacteria using G-spin™ Genomic DNA extraction Kit (iNtRON Biotechnology, Cat. No. 17121), then subjected to amplification. For the genomic DNA extraction, 500 mL of the bacterial suspension was centrifuged at 13,000 rpm for 1 min and the supernatant was removed then, 500 mL of PBS (pH 7.2) was added thereto, followed by centrifuging to remove supernatant. Then, cell pellets were suspended by adding 300 mL of CJ-buffer solution containing RNase A and Proteinase K, and left to stand at 65° C. for 15 min, then 250 mL of binding buffer solution was added thereto to mix thoroughly, followed by binding DNA to a spin column. After that, 500 mL of washing buffer A was added to the spin column and centrifuged at 13,000 rpm for 1 mm to wash, and 500 mL of washing buffer B was added to the spin column to centrifuge, then the column was moved to a 1.5 mL microcentrifuge tube, followed by adding 50 mL of elution buffer to centrifuge for 1 min, thus obtaining 15.8 ng/mL genomic DNA. The obtained genomic DNA was diluted in a given ratio and used as a template of isothermal amplification reaction.

An external primer (SEQ ID NO: 1 and SEQ ID NO: 2) was designed such that it comprises sequences complementary to the Chlamydia trachomatis cryptic plasmid DNA.

| | |
|---|---|
| SEQ ID NO: 1: | 5′-TAAACATGAAAACTCGTTCCG-3′ |
| SEQ ID NO: 2: | 5′-TTTTATGATGAGAACACTTAAACTCA-3′ |

A DNA-RNA-DNA hybrid primer (SEQ ID NO: 3 and SEQ ID NO: 4) was designed such that the 5′-end of oligo DNA-RNA region thereof has a sequence non-complementary to Chlamydia trachomatis cryptic plasmid DNA, and the 3′-end of oligo DNA region thereof has a sequence complementary to Chlamydia trachomatis cryptic plasmid DNA (oligo RNA regions are underlined).

SEQ ID NO: 3:
5′-ATTCACCGCATCGAATCGATGTAAAATAGAAAATCGCATGCAAGAT
A-3′
SEQ ID NO: 4:
5′-TATCGATTCCGCTCCAGACTTAAAAAGCTGCCTCAGAATATACTCA
G-3′

A DNA-RNA-DNA hybrid signal probe (SEQ ID NO: 5) for performing signal amplification, has a base sequence complementary to DNA amplified by the above primer set, and is labeled with fluorescein and biotin at the 5′-end and the 3′-end thereof, respectively (oligo RNA region is underlined):

SEQ ID NO: 5:
5′-Fluorescein-GCTTTGTTAGGTAAAGCTCTGATA
TTTG-biotin-3′

In order to amplify target nucleic acids using the external primer set and hybrid primer set, a reaction mixture containing the external primer set, the hybrid primer set and target DNA was prepared, 10 mM of (NH₄)₂SO₄, 4 mM of MgSO₄, 10 nM of KCl, 0.25 nM of each dNTP (Fermentas), 2.9 mM of DTT, 0.1 μg of BSA, 0.1 mM spermine, 0.05 mM EGTA, 0.1 μM of external primer set, 0.5 μM of inner primer set and 10 fg˜1 ng of Chlamydia trachomatis cryptic plasmid DNA were added to 10 mM of Tris-HCl (pH 8.5) buffer to prepare the reaction mixture.

The reaction mixture was denatured for 5 min at 95° C. cooled for 5 min at 60° C., and added with an enzymatic reaction mixture solution to a final volume of 20 μl for DNA amplification, followed by carrying out isothermal amplification for 1 hr at 60° C.

The composition of enzymatic reaction mixture solution is as follows: 0.3 μg of T4 Gene 32 protein (USB), 6 units of RNase inhibitor (Intron), 3 unit of RNaseH (Epicentre), 6 units of Bst DNA polymerase (NEBM0275M) and 1 nM DNA-RNA-DNA hybrid signal probe.

Meanwhile, human genomic DNA was used as a control. 6 μl of reaction solution was taken after the amplification reaction, and mixed with a loading buffer, then subjected to electrophoresis on 1.8% agarose gel containing ethidium bromide, followed by determining amplification efficiency with band visualization on a UV transilluminator.

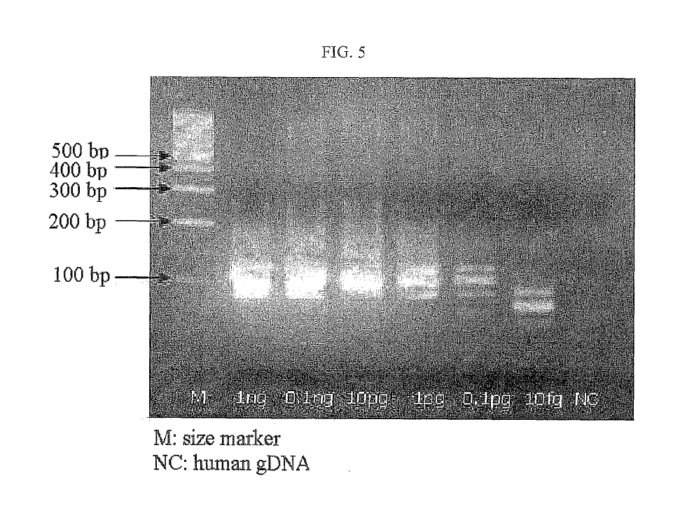

As a result, as shown in FIG. 5, it was confirmed that target DNA amplification product was present in the sample added with Chlamydia trachomatis cryptic plasmid DNA, compared with the sample added with human genomic DNA.

### Example 2

### DNA Detection by Enzyme-Immunoassay

170 ml MST binding buffer was added to the amplification product obtained in Example 1 to prepare a reaction mixture consisting of the following components: 136 mM of NaCl, 2.7 mM of KCl, 8.1 mM of Na₂HPO₄, 1.5 mM H₂PO₄, 0.05% Tween 20, 1/7000 diluted anti-F-HRP (Perkin Elmer, horseradish peroxidase conjugated anti-fluorescent antibody). The reaction mixture was transferred to streptavidin-coated microplate wells (Roche), and allowed to react for 10 min at 37° C. and 200 rpm. The supernatant in each well was removed and each well was added with 300 ml of PBST washing buffer to wash, wherein the PBST washing buffer has the same composition as that of the above binding buffer except for the antibody removed therefrom. After washing, each well was added with 200 ml of HRP substrate, 3,3′,5,5′-tetramethylbenzidine (Bio-Rad, TMB), and incubated for 5 min in a dark place to result in color development, then added with 100 ml of 1N H₂SO₄ to stop the reaction. In order to determine the effectiveness of the sample and the control, the absorbance values at 465 nm were compared using an ELISA reader (Zenyth 340rt). It is determined that the larger the difference between the values is, the more effective it is.

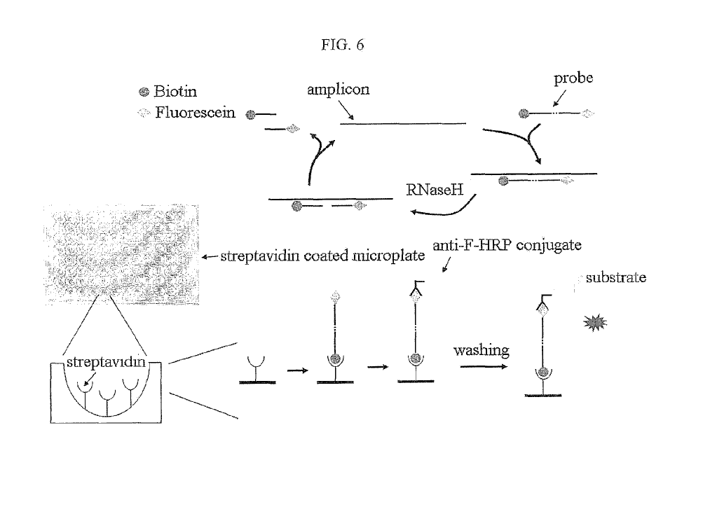

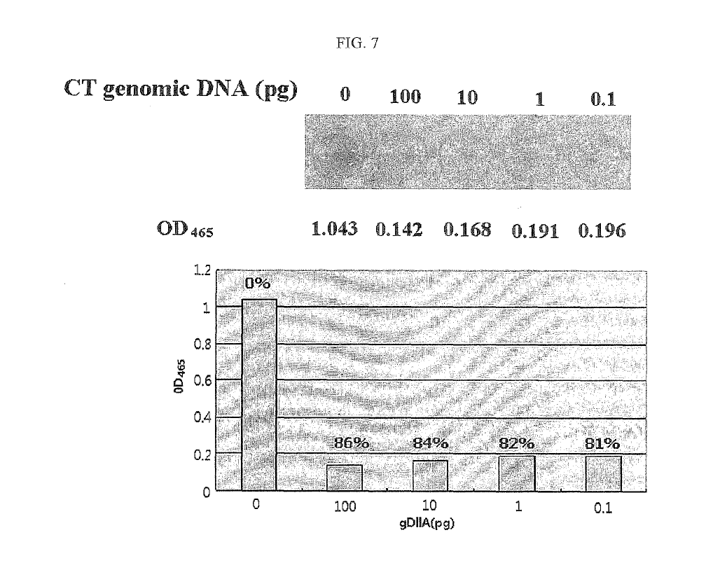

As a result, as shown in FIG. 6 and FIG. 7, it was confirmed that the experimental sample with the amplification product did not result in color development by HRP (horseradish peroxidase) conjugated with anti-fluorescein.

### Example 3

### DNA Detection by Lateral-Flow Chromatography

10 ml gold colloid solution (Chemicon) with a diameter of 40 nm was added to 100 mg streptavidin (Sigma), and vortexed for 2 min, then allowed to react for 3 hr. Then, 1 mL of 1% BSA (dissolved in 2 mM borate) solution was added to the resulting mixture to centrifuge at 10,000 rpm for 15 min at 4° C. and supernatant was removed, then 1 mL of 2 mM borate buffer solution was added to the resultant from which the supernatant was removed to wash 3 times, followed by adding 1% BSA (dissolved in 2 mM borate) to re-suspend.

Gold conjugate solution was stored at 4° C. with an absorbance value of 10 at 520 nm, and used by diluting to an appropriate ratio. Fluorescein antibody (Chemicon) was coated as a test line and biotin-conjugated casein (Biofocus) was coated as a control line on a nitrocellulose membrane, respectively.

60 mL of gold conjugate solution diluted 1:50 with a running buffer (1×PBS, 1% Triton X-100, 0.6% BSA) was added to the amplification product obtained in Example 1, and nitrocellulose membrane strip was soaked into the solution and subjected to lateral flow chromatography for 10 min at room temperature, thus detecting the existence of target nucleic acids by examining of the test line.

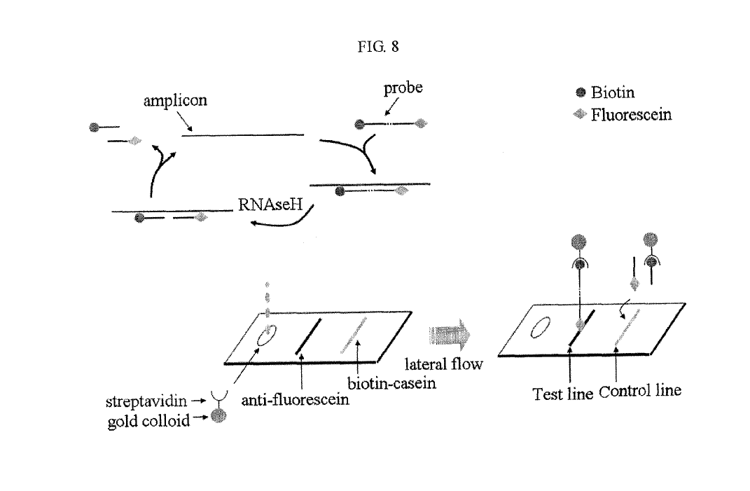

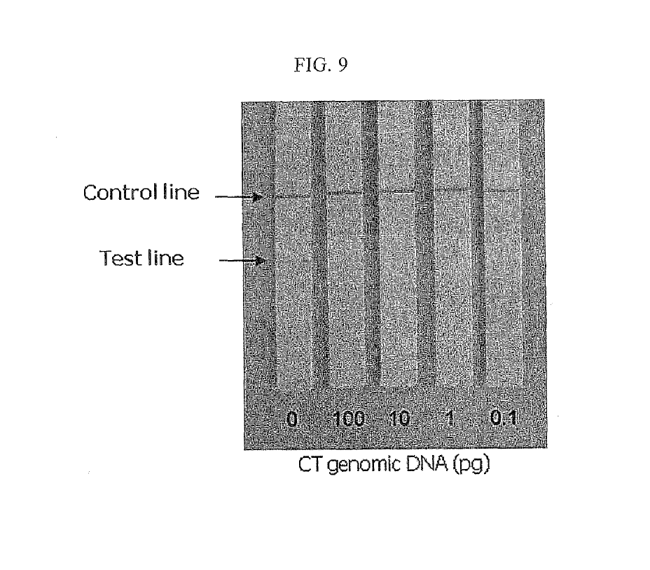

As a result, as shown in FIG. 8 and FIG. 9, in the negative control sample, two lines appeared (test line and control line), whereas, in the sample added with Chlamydia trachomatis cryptic plasmid DNA, only one line (control line). Thus, it could be confirmed that target nucleic acid amplification product is present in the test sample.

### Example 4

### Isothermal Amplification of RNA

RNA transcribed in vitro from plasmid DNA having cDNA of Norovirus G1 Type RNA cloned into pDrive vector, was used as a target RNA. MEGAscript High Yield Transcription kit (Ambion, Cat. No. AMI 333) was used to perform in vitro transcription. In vitro transcription reaction was performed as follows; a plasmid DNA template was linearized using a restriction enzyme, and 1 mg of DNA as added with 8 mL of dNTP (dATP, dUTP, dGTP, dCTP) mixture, 2 mL of 10× reaction buffer, mL of T7 RNA polymerase and nuclease-free water to a final volume of 20 mL to mix thoroughly, then allowed to react for 4 hr at 37° C. After completion of the reaction, in order to remove the DNA template, 1 mL of Turbo

DNase was added to the resulting mixture and allowed to react at 37° C. for 15 min then the amplified RNA was purified by RNeasy MinElute Cleanup Kit (Qiagen, Cat. No. 74204). The purified RNA was diluted in a given ratio and used as a template for isothermal amplification reaction.

An external primer (SEQ ID No. 6 and SEQ ID No. 7) was designed such that it comprises sequences complementary to the Norovirus G1 Type RNA.

| | |
|---|---|
| SEQ ID NO: 6: | 5′-ATGCGGTTCCACGATCTTGG-3′ |
| SEQ ID NO: 7: | 5′-GCGACTGCTGTTGAATCACC-3′ |

A DNA-RNA-DNA hybrid primer (SEQ ID NO: 8 and SEQ ID NO: 9) was designed such that the 5′-end of oligo DNA-RNA region thereof has a sequence non-complementary to Norovirus G1 Type RNA, and the 3′-end of oligo DNA region thereof has a sequence complementary to Norovirus G1 Type RNA (oligo RNA regions are underlined).

SEQ ID NO: 8:
5′-CCAATTCACAAGTGAAGAGCAAAATCTCCTGCCCGAATTCGTAA-3′
SEQ ID NO: 9:
5′-TCTACCGCTGATCATGTGCTAAAATGCTCAGCTGTATTAGCCTC-3′

A DNA-RNA-DNA hybrid signal probe (SEQ ID NO: 10) for performing signal amplification, has a base sequence complementary to DNA amplified by the above primer set, and is labeled with fluorescein and biotin at the 5′-end and the 3′-end thereof, respectively (oligo RNA region is underlined):

SEQ ID NO: 10:
5′-Fluorescein-GCCCGAATTCGTAAAUGATGATGGCGTC-
biotin-5′

In order to amplify target RNA using the external primer set, the hybrid primer set and the hybrid signal probe, a reaction mixture was prepared. 10 mM of (NH₄)₂SO₄, 16 of MgSO₄, 10 mM of KCl, 0.25 mM of each dNTP (Fermentas), 2.9 mM of DTT, 0.1 μg of BSA, 0.1 μM of external primer set, 0.5 μM of hybrid primer set, 0.3 μg T4 Gene 32 Protein (USB), 10 unit RNase inhibitor (Intron), 9 unit RNaseH (Epicentre), 3 unit Bst DNA polymerase (NEM0275M), 3 unit AMV reverse transcriptase (USB), 10 nM DNA-RNA-DNA hybrid signal probe and 100 pg Norovirus G1 Type RNA were added to 10 mM of Tris-HCl (pH 8.5) buffer to a final volume of 20 μl, thus preparing the reaction mixture. The reaction mixture was subjected to isothermal amplification at 60° C.; for 90 min.

Meanwhile, human RNA was used as a control. 6 μl of reaction solution was taken after the amplification reaction, and mixed with a loading buffer, then subjected to electrophoresis on 2.5% agarose gel containing ethidium bromide, followed by determining amplification efficiency with band visualization on a UV transilluminator.

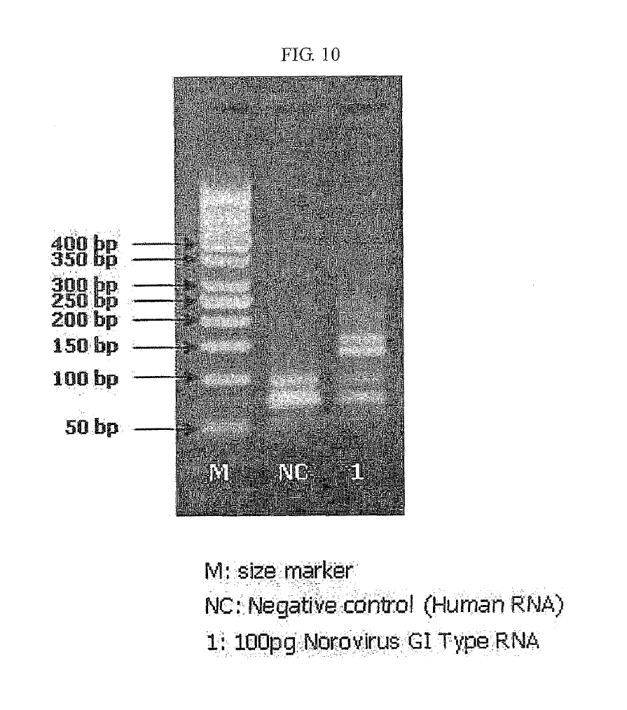

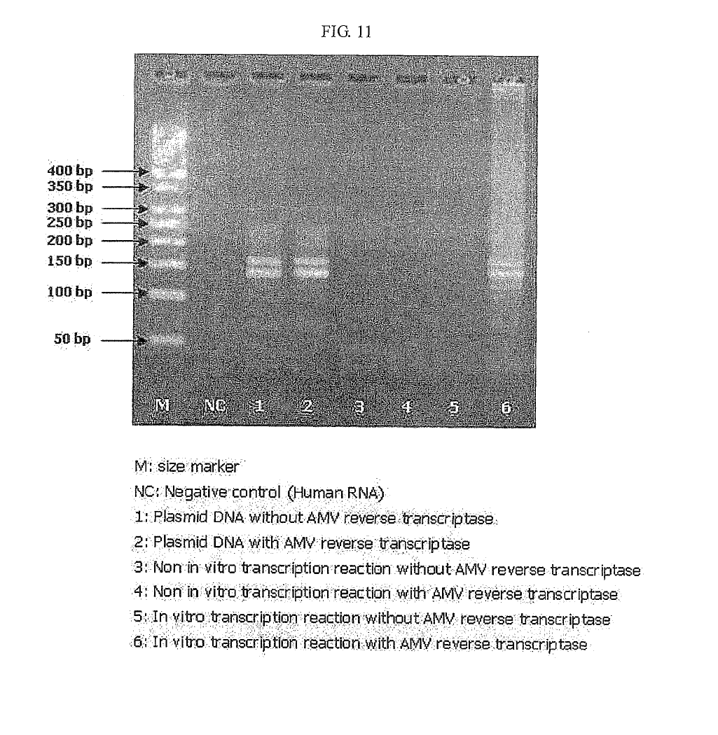

As a result, as shown in FIG. 10, it was confirmed that target DNA amplification product was present in the sample added with Norovirus G1 Type RNA, compared with the negative control added with human RNA. In addition, in order to examine whether the amplification product was resulted from DNA contamination, the same experiment as described above was performed using Norovirus G1 Type plasmid DNA and the enzymatic reaction mixture solution with and without AMV reverse transcriptase. As a result, as shown in FIG. 11, it was confirmed that the amplified product was an amplification product obtained by amplifying RNA as a template.

### Example 5

### RNA Detection by Enzyme-Immunoassay

170 ml of PEST binding buffer was added to the amplification product obtained in Example 4 to prepare a reaction mixture consisting of the following components: 136 mM Of NaCl, 2.7 mM of KCl, 8.1 mM of Na₂HPO₄, 1.5 mM KH₂PO₄, 0.05% Tween 20, 1/7000 diluted anti-F-HRP (Perkin Elmer, horseradish peroxidase conjugated anti-fluorescent antibody). The reaction mixture was transferred to streptavidin-coated microplate wells (Roche), and allowed to react for 10 min at 37° C. and 200 rpm. The supernatant in each well was removed and each well was added with 300 ml of PBST washing buffer to wash, wherein the PBST washing buffer has the same composition as that of the above binding buffer except for the antibody removed therefrom. After washing, each well was added with 200 ml of HRP substrate, 3,3′,5,5′-tetramethylbenzidine (Bio-Rad, TMB), and incubated for 5 min in a dark place to result in color development, then added with 100 ml of IN H₂SO₄ to stop the reaction. In order to determine the effectiveness of the sample and the control, the absorbance values at 465 nm were compared using an ELISA reader (Zenyth 340rt). It is determined that the larger the difference between the values is, the more effective it is.

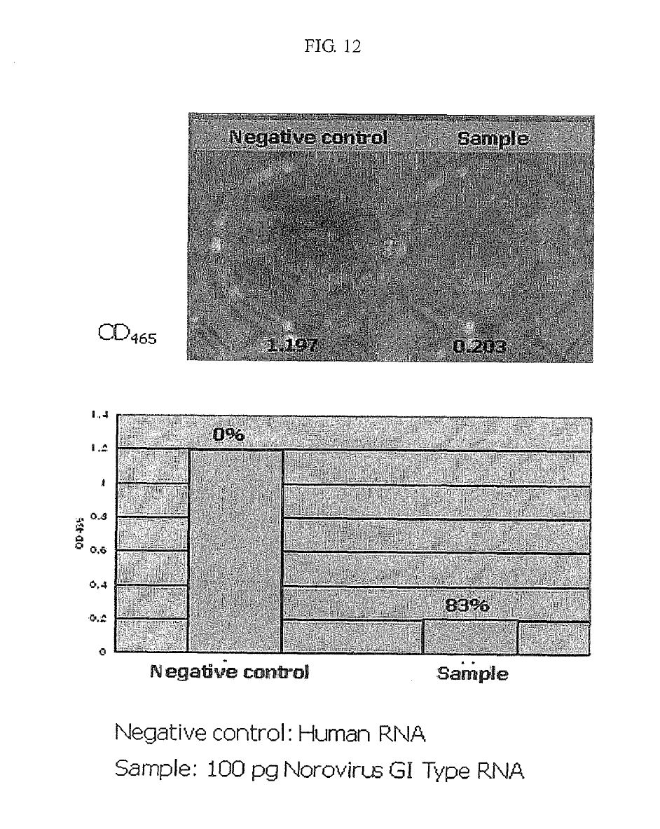

As a result, as shown in FIG. 12, it was confirmed that the experimental sample with the amplification product did not result in color development by HRP (horseradish peroxidase) conjugated with anti-fluorescein.

### INDUSTRIAL APPLICABILITY

As described above in detail, an embodiment of the present invention provides a method for amplifying target nucleic acids rapidly and exactly at isothermal temperature, and a method for detecting nucleic acids, which comprises simultaneously performing amplifications of target nucleic acids and a signal probe at isothermal temperature. The method according to an embodiment of the present invention can be used to amplify target nucleic acids in a sample, rapid and exact manner without the risk of contamination compared to the conventional methods such as PCR, and it can simultaneously amplify target nucleic acid and a signal probe, so that it can be applied to various genome projects, detection and identification of a pathogen, detection of gone modification producing a predetermined phenotype, detection of hereditary diseases or determination of sensibility to diseases, and estimation of gene expression. Thus, it is useful for molecular biological studies and disease diagnosis.

Although the present invention has been described in detail with reference to the specific features, it will be apparent to those skilled in the art that this description is only for a preferred embodiment and does not limn the scope of the present invention. Thus, the substantial scope of the present invention will be defined by the appended claims and equivalents thereof.

What is claimed:

1. A method for isothermal amplification of target DNA, the method comprising the steps of:
(a) denaturing a reaction mixture containing (i) target DNA, (ii) an external primer set having a base sequence complementary to the target DNA, and (iii) a DNA-RNA-DNA hybrid primer set having a base sequence complementary to the target DNA at the 3′-terminal end and non-complementary to the target DNA at the 5′-terminal end, wherein the DNA-RNA-DNA hybrid primer set consists of 44˜66 bases in length, the 5′-DNA region of the DNA-RNA-DNA hybrid primer is 20˜30 basis in length, the RNA region of the DNA-RNA DNA hybrid primer is 4˜6 bases and the 3′-DNA of the DNA-RNA-DNA hybrid primer is 20˜30 bases in length; and
(b) adding an enzymatic reaction mixture solution containing RNase, DNA polymerase capable of performing strand displacement and a DNA-RNA-DNA hybrid signal probe having a base sequence complementary to the amplification product produced by the external primer set and the hybrid primer set, to the reaction mixture denatured in the step (a), wherein the DNA-RNA-DNA hybrid signal probe consists of 24˜36 bases in length and the RNA portion located in the middle thereof consists of 4˜6 bases in length, and then simultaneously amplifying said target DNA and said signal probe at isothermal temperature.

2. The method for isothermal amplification of target DNA according to claim 1, wherein the external primer set is any one selected from the group consisting of oligo DNA, oligo RNA, and hybrid oligo RNA/DNA.

3. The method for isothermal amplification of target DNA according to claim 1, wherein the DNA-RNA-DNA hybrid primer set is non-complementary to a target DNA at the 5′-end of DNA-RNA, and complementary to the target DNA at the 3′-end of DNA.

4. The method for isothermal amplification of target DNA according to claim 1, wherein the DNA polymerase is a thermostable DNA polymerase with no exonuclease activity.

5. The method for isothermal amplification of target DNA according to claim 1, wherein the RNase is RNase H.

6. The method for isothermal amplification of target DNA according to claim 1, wherein the DNA-RNA-DNA hybrid signal probe is labeled with markers at the end thereof.

7. The method for isothermal amplification of target DNA according to claim 7, wherein the markers are selected from the group consisting of biotin, fluorescein, digoxygenin, and 2,4-dinitrophenyl.

8. The method for isothermal amplification of target nucleic acids according to claim 1, wherein the isothermal amplification is carried out at a temperature of 60˜70° C.

9. A method for isothermal amplification of target RNA, the method comprising the steps of:
adding a reaction mixture containing (i) target RNA, (ii) an external primer set having a base sequence complementary to the target RNA, and (iii) a DNA-RNA-DNA hybrid primer set having a base sequence complementary to the target RNA at the 3′-terminal end and non-complementary to the target RNA at the 5′-terminal end, wherein the DNA-RNA-DNA hybrid primer set consists of 44˜66 bases in length, the 5′-DNA region of the DNA-RNA-DNA hybrid primer is 20˜30 basis in length, the RNA region of the DNA-RNA-DNA hybrid primer is 4˜6 bases and the 3′-DNA of the DNA-RNA-DNA hybrid primer is 20˜30 bases in length, to an enzymatic reaction mixture solution containing (iv) DNA polymerase capable of performing strand displacement, RNase, reverse transcriptase and a DNA-RNA-DNA hybrid signal probe having a base sequence complementary to the amplification product produced by the external primer set and the hybrid primer set, wherein the DNA-RNA-DNA hybrid signal probe consists of 24˜36 bases in length and the RNA portion located in the middle thereof consists of 4˜6 bases in length, and then simultaneously amplifying said target RNA and said signal probe at isothermal temperature.

10. The method for isothermal amplification of target RNA according to claim 9, wherein the external primer set is any one selected from the group consisting of oligo DNA, oligo RNA, and hybrid oligo RNA/DNA.

11. The method for isothermal amplification of target RNA according to claim 9, wherein DNA-RNA-DNA hybrid primer set is non-complementary to a target RNA at the 5′-end of DNA-RNA, and complementary to the target RNA at the 3′-end of DNA.

12. The method for isothermal amplification of target RNA according to claim 9, wherein the DNA polymerase is a thermostable DNA polymerase with no exonuclease activity.

13. The method for isothermal amplification of target RNA according to claim 9, wherein the RNase is RNase H.

14. The method for isothermal amplification of target RNA according to claim 9, wherein the reverse transcriptase is AMV (avian myelobalstosis virus) reverse transcriptase or MMLV (maloney murine leukemia virus) reverse transcriptase.

15. The method for isothermal amplification of target RNA according to claim 9, wherein the DNA-RNA-DNA hybrid signal probe is labeled with markers at the end thereof.

16. The method for isothermal amplification of target RNA according to claim 15, wherein the markers are selected from the group consisting of biotin, fluorescein, digoxygenin, and 2,4-dinitrophenyl.

17. The method for isothermal amplification of target RNA according to claim 9, wherein the isothermal amplification is carried out at a temperature of 60˜70° C.
